(19) United States
(12) Patent Application Publication
(10) Pub. No.: US 2023/0025555 A1
(43) Pub. Date: Jan. 26, 2023

(54) PRE-FILLED SYRINGE STOPPER RETAINER HAVING PROJECTIONS OR RECEIVERS AND METHODS OF USING SAME

(71) Applicant: Merck Sharp & Dohme LLC, Rahway, NJ (US)

(72) Inventors: Ophelia Wells, Easton, PA (US); Wail Rasheed, Union, NJ (US); Raymond K. Seto, Warren, NJ (US); Cassie Megna, New Milford, NJ (US); Eduardo Cristofolli, Elizabeth, NJ (US); Michael F. Tormey, Princeton Junction, NJ (US)

(73) Assignee: Merck Sharp & Dohme LLC, Rahway, NJ (US)

(21) Appl. No.: 17/782,934

(22) PCT Filed: Dec. 11, 2020
(86) PCT No.: PCT/US2020/064435

§ 371 Date: Jun. 6, 2022

Related U.S. Application Data
(60) Provisional application No. 62/948,550, filed on Dec. 16, 2019.

(51) Int. Cl. A61M 5/00; A61M 5/315; B65D 25/10

(52) U.S. Cl. CPC A61M 5/008; A61M 5/31515; B65D 25/108

(57) ABSTRACT

A stopper retainer comprises a base having an upper surface, a lower surface, and a perimeter; a sidewall joined to the base around the perimeter, the sidewall having a raised height to define an interior cavity with the base; and a plurality of projections extending from the lower surface of the base, each of the plurality of projections being sized to at least partially fit within a lumen of a syringe barrel.

### FIELD OF THE INVENTION

The present disclosure relates generally to storing and transporting syringes. More specifically, the present disclosure relates to a closure device and related methods that allow for safer storage and transportation of pre-filled syringes.

### BACKGROUND OF THE INVENTION

A pre-filled syringe typically includes a glass barrel containing a pharmaceutical product, which is sealed by a stopper. The stopper creates a sterile barrier between the product and the external environment, and is designed to maintain container closure integrity through precise contact with the glass barrel. Gas bubbles within the pre-filled syringe can expand or contract in response to changes in ambient pressure, causing the stopper in the syringe to move from its original position. This movement of the stopper may encourage contamination ingress and the loss of sterility of the product.

Thus, there exists a need for devices that improve upon and advance the methods of safely storing and transporting syringes, such as pre-filled syringes.

### SUMMARY OF THE INVENTION

In one embodiment, a stopper retainer comprises a base having an upper surface, a lower surface, and a perimeter; a sidewall joined to the base around the perimeter, the sidewall having a raised height to define an interior cavity with the base; and a plurality of projections extending from the lower surface of the base, each of the plurality of projections being sized to at least partially fit within a lumen of a syringe barrel.

In another embodiment, the above stopper retainer further comprises a plurality of depressions formed in the upper surface of the base and disposed in rows and columns.

In another embodiment of the above stopper retainer, the plurality of projections comprises a first number of projections, and the plurality of depressions comprises a second number of depressions, the first number and the second number being equal.

In another embodiment of the above stopper retainer, each of the plurality of depressions is aligned with one of the plurality of projections.

In another embodiment of the above stopper retainer, the plurality of projections is sized to at least partially fit within the plurality of depressions.

In one embodiment, a system of storing syringes comprises a tub having an interior void; a first tray having a plurality of apertures; and the stopper retainer described above; wherein each of the apertures of the first tray is directly aligned with one of the plurality of projections of the stopper retainer.

In another embodiment, the above system further comprises a second tub and a second tray having a plurality of second apertures, wherein the second tub and the second tray are disposed on top of the stopper retainer.

In another embodiment, the above system further comprises a second stopper retainer, wherein each of the apertures of the second tray are directly aligned with one of the plurality of projections of the second stopper retainer.

In another embodiment, the above system further comprises 1-100 additional tubs and trays, and 1-100 additional stopper retainers, wherein each of the apertures of each additional tray are directly aligned with one of the plurality of projections of the additional stopper retainer immediately on top of the additional tray.

In one embodiment, a stopper retainer comprises a body having an upper surface and a lower surface, and a plurality of receivers disposed on the lower surface of the body, each of the plurality of receivers being sized to fit a portion of a plunger rod thumb press.

In another embodiment of the above stopper retainer, each of the plurality of receivers is circular, square or irregular in shape.

In another embodiment of the above stopper retainer, each of the plurality of receivers comprises a circular, square or irregular shaped indentation in the body.

In another embodiment of the above stopper retainer, each of the plurality of receivers comprises a protruding circular, square or irregular shaped rim on the body.

In another embodiment, the above stopper retainer further comprises at least one arm extending from the lower surface of the body.

In one embodiment, a system of storing syringes comprises a tub having an interior void; a first tray having a plurality of apertures; and a stopper retainer described above; wherein each of the apertures of the first tray is directly aligned with one of the plurality of receivers of the stopper retainer.

In another embodiment, the above system further comprises a plurality of syringes, each of the plurality of syringes comprising a barrel and a plunger rod, and each plunger rod being at least partially disposed in one of the plurality of receivers.

### BRIEF DESCRIPTION OF THE DRAWINGS

Various embodiments of the presently disclosed stopper retainers are disclosed herein with reference to the drawings, wherein:

FIGS. 1A-B are schematic illustrations of a pre-filled syringe;

FIGS. 2A-B are schematic illustrations showing a possible movement of a stopper during transportation;

FIGS. 3A-B are schematic perspective and top views of a stackable retainer according to another embodiment of a retainer;

FIG. 3C is a schematic side view of several examples of projections having different shapes;

FIG. 3D is a schematic cross-sectional view of one example of a base having a depression over a projection;

FIGS. 4A-B are schematic perspective views showing the use of a stackable retainer similar to that of FIG. 3A;

FIGS. 5A-C are schematic top, upper perspective, and lower perspective views of a retaining cover according to another embodiment of a retainer; and

FIGS. 6-7 are a photograph and a cross-sectional view showing the use of a retainer of FIGS. 5A-C.

Various embodiments will now be described with reference to the appended drawings. It is to be appreciated that these drawings depict only some embodiments of the disclosure and are therefore not to be considered limiting of its scope.

### DETAILED DESCRIPTION OF THE INVENTION

Despite the various improvements that have been made to storage and transporting solutions for pre-filled syringes, conventional methods suffer from some shortcomings as discussed above.

There, therefore, is a need for further improvements to the devices and methods used to help facilitate proper and safer transportation and/or storage of pre-filled syringes. Among other advantages, the present disclosure may address one or more of these needs.

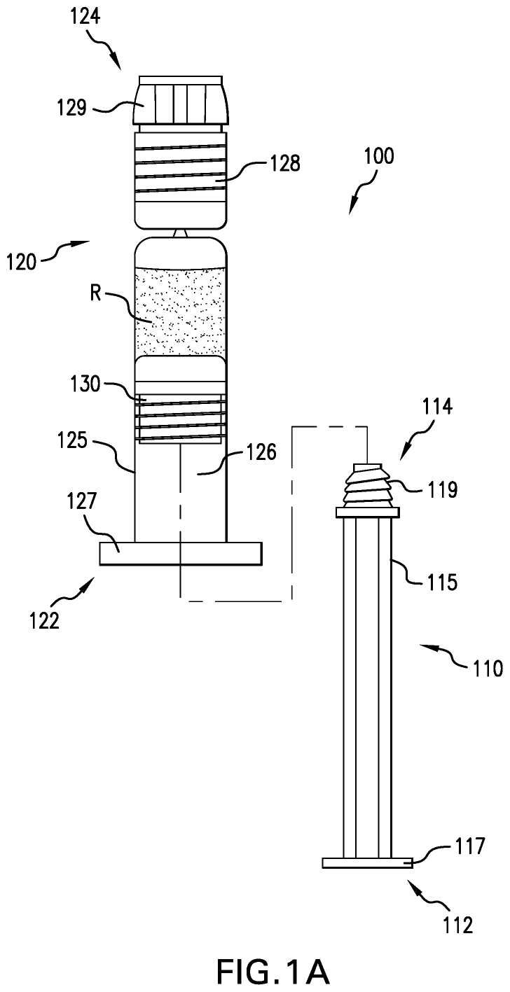

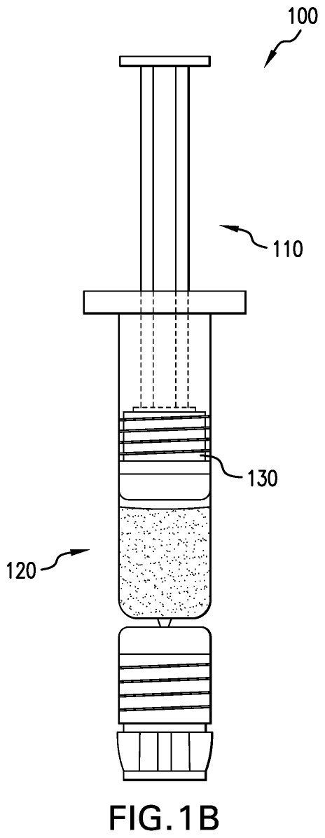

Reference is now made to FIGS. 1A-B, which show schematic illustrations of a prefilled-syringe 100. Pre-filled syringe 100 generally comprises two main portions, a plunger rod 110 and a barrel 120. Plunger rod 110 generally extends between a proximal end 112 and a distal end 114, and comprises an elongated piston 115 extending between a thumb press 117 and a coupler 119. In one embodiment, piston 115 has a cruciform cross-sectional shape.

A cylindrical barrel 120 extends between proximal end 122 and distal end 124 and comprises a body 125 defining a lumen 126 for accepting a portion of plunger rod 110. Body 125 further comprises a flange 127 adjacent proximal end 122, and a removable cap 129 mateable with a hub 128 adjacent distal end 124. Body 125 defines a reservoir “R” that holds a medicament, drug, saline, or other substance. An internally threaded stopper 130 is disposed inside lumen 126 of body 125. In one embodiment, stopper 130 is made of an elastomeric material such as natural rubber, synthetic rubber, thermoplastic elastomers, or combinations thereof, and comprises an opening to receive and mate with coupler 119 of plunger rod 110 by advancing the plunger rod inside the barrel lumen 126 and rotating at least one of coupler 119 and stopper 130 relative to the other. The barrel 120 and the plunger rod 110 may be assembled as shown in FIG. 1B.

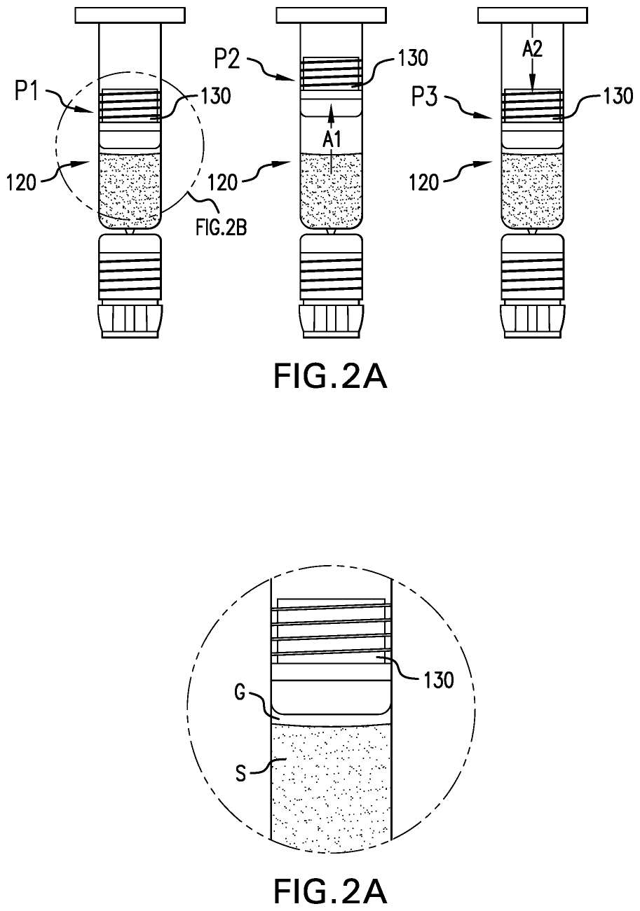

FIGS. 2A-B illustrate how a stopper 130 can move within a barrel 120. When assembled, stopper 130 may be disposed within barrel 120 at position “P1” at ambient temperature and pressure. A gas “G” may be located between the substance “S” and stopper 130. If, during transportation, the barrel is brought to an area of reduced pressure, the gas “G” may expand and stopper 130 may move upward within the barrel in the direction of arrow A1 to position “P2”. This may occur, for example, if the barrel is transported to a location with high elevation and lower atmospheric pressure. If the barrel is subsequently returned to the original pressure, the stopper may move downward within the barrel in the direction of arrow A2 to position “P3”. The movement of the stopper within the barrel may result in contamination ingress and the loss of sterility of the product as previously noted.

Pre-filled syringes 100 are typically stored and transported in lots disposed in tubs. Specifically, tubs may comprise a body defining a shoulder forming a perimeter and an interior void having a depth capable of accommodating at least a major portion of a syringe barrel. In some embodiments, a tub has a generally square or rectangular transverse cross-section and may accommodate between 1 and 200 syringes.

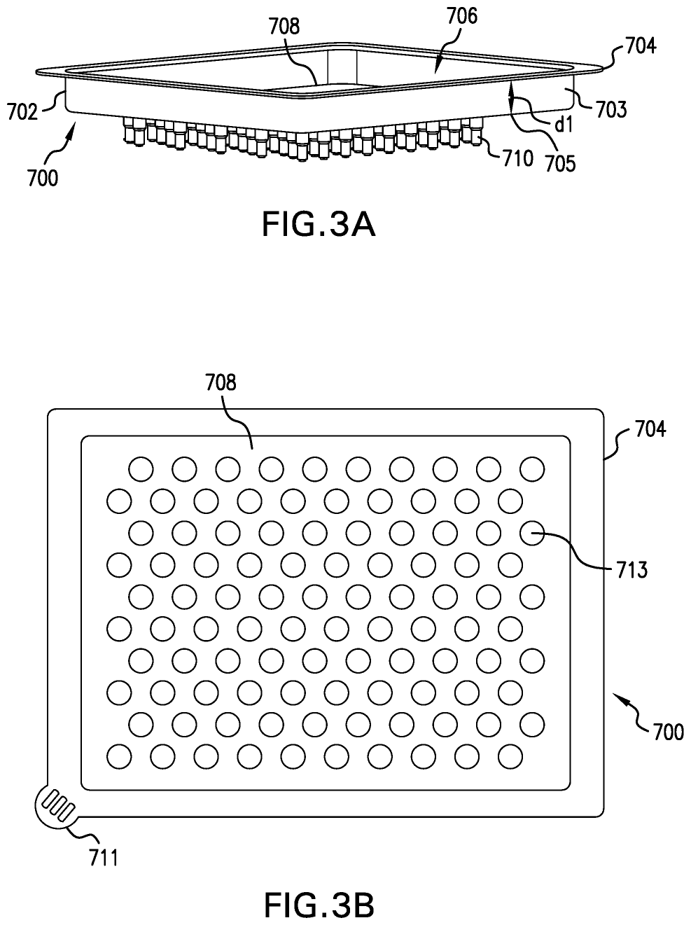

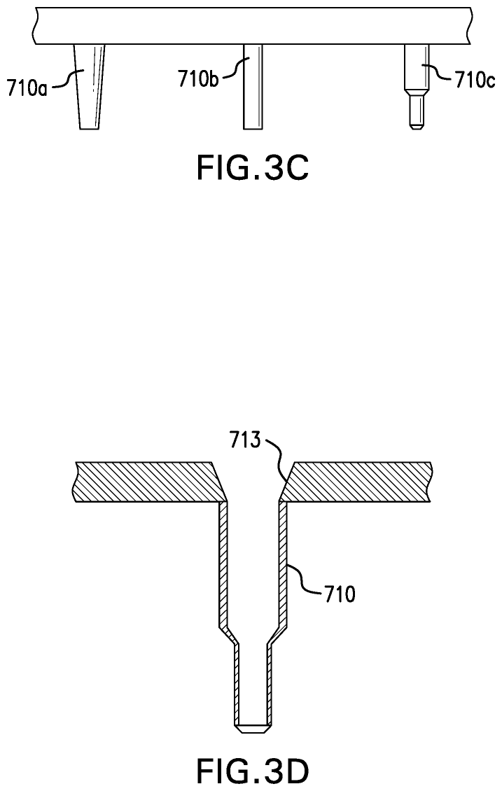

In some embodiments, a stackable retainer 700 is used to impede the movement of stoppers of syringes disposed within a tub. FIGS. 3A-B are schematic perspective and top views of a stackable retainer. Stackable retainer 700 generally comprises a rectangular or square-shaped body 702 having a sidewall 703 and a shoulder 704, and defining an interior cavity 706 formed by the sidewall 703 to base 708. Shoulder 704 may include a flap 711 at one corner to aid in assembly and/or removal of the device. Base 708 may comprise a series of depressions 713. The interior cavity 706 may have a depth “dl”, which is approximately the height of the sidewall, of between ½ inch to 4 inches.

In some embodiments, a number of projections 710 may extend from the lower surface 705 of base 708. Each projection may be approximately ¼ inch to 1 inch in length and between 1/16 inch and 1 inch in diameter. Each projection may be sized to fit within lumen 126 of a barrel 120 and extend toward the stopper 130 disposed within the barrel 120 to make contact with the stopper 130 or terminate immediately adjacent the stopper 130. The length of the projections 710 may also be dependent on the size of the barrel 120 and/or the quantity of the substance within the barrel 120.

Various shapes of the projections are possible. FIG. 3C illustrates three variations of the projections. In some embodiments, the projections 710a are cone-shaped (i.e., each projection has a decreasing diameter from lower surface 705 toward their distal ends farthest from the lower surface). Instead of being cone-shaped, the retainer may comprise cylindrical projections 710b and have a constant diameter along their height. In a third embodiment, step-shaped projections 710c may include a pair of cylindrical portions with tapered cones forming steps therebetween. It will be understood that all of the projections on a given retainer may be the same, or may include combinations of the shapes described.

The projections 710 may be arranged in rows and columns to reflect the arrangement of the barrels within the tray and the projections may align with the apertures of the tray. In some embodiments, between 2 and 20 rows and between 2 and 20 columns of projections are formed on the lower surface of body 702. In some embodiments, each of the rows of projections may be offset from adjacent rows. In some embodiments, each of the columns of projections may be offset from adjacent columns. Offsetting of rows and/or columns may allow for an increase in the density of the projections and the number of barrels to be transported in a given tub.

In one embodiment, projections 710 are step-shaped. The number and location of projections may be varied as desired, and may match the number of apertures in a tray with which the retainer will be used. As shown in the top view of stackable retainer 700 (FIG. 3B), base 708 may comprise a series of depressions 713. In some embodiments, each depression 713 is longitudinally aligned with an underlying projection 710 (FIG. 3D). In some embodiments, the depression 713 extends at least partially into the underlying projection 710.

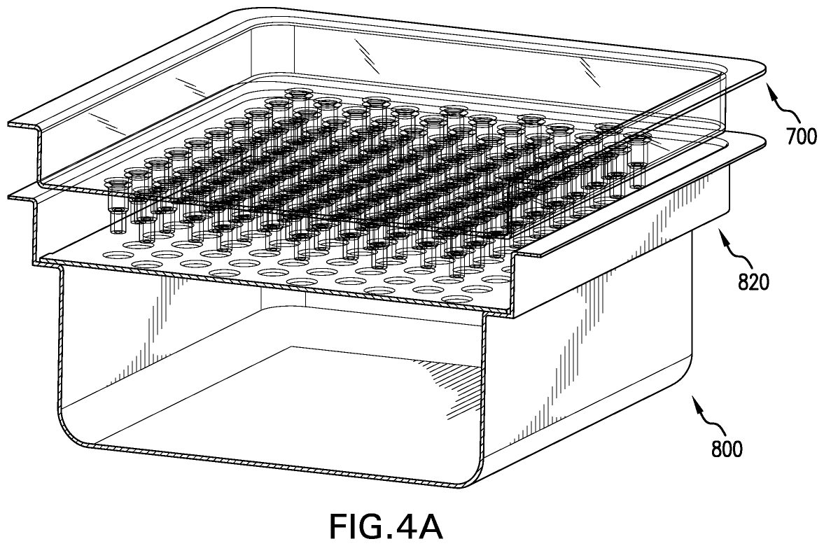

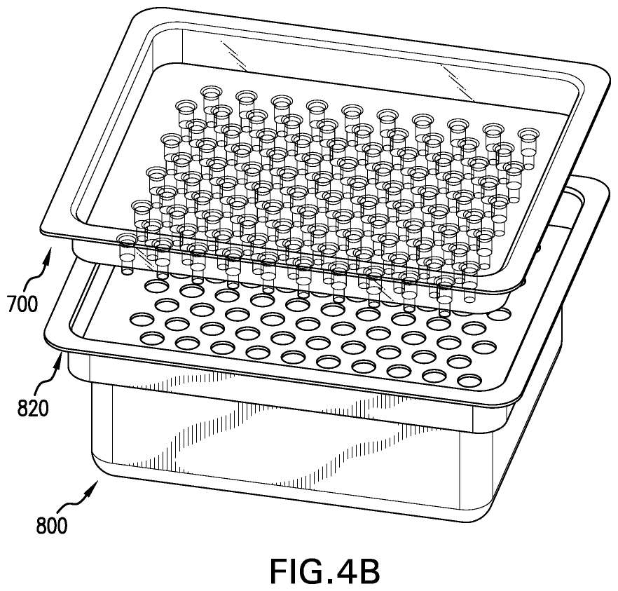

FIGS. 4A-B show embodiments where a stackable retainer 700 is used with tub 800 and tray 820. The three components may be arranged with tub 800 at the bottom, a tray 820 having a plurality of syringe barrels (not shown) and the stackable retainer above the tray. Projections 710 of stackable retainer 700 may extend into each of the barrels. Specifically, each projection 710 extends from lower surface 705 of the body 702, and enters a lumen 126 of barrel 120, terminating adjacent stopper 130 within the lumen of the barrel. Projection 710 may contact the stopper 130 or may terminate immediately adjacent the stopper (e.g., within ⅛ of an inch from the stopper or some other specific tolerance). With projections 710 being disposed in each of the barrels 120, the projections will prevent the stoppers from moving upward upon the expansion of gas when the pressure is reduced, reducing the risk of contamination and/or loss of sterility. Moreover, with stackable retainer 700 being disposed above the tray, interior cavity 706 is available to accept a second tub 800 having a second tray 820 having another quantity of syringe barrels, and a second retainer may be disposed above the second tray. In this manner, multiple units of tubs-trays-retainers may be stacked upon one another.

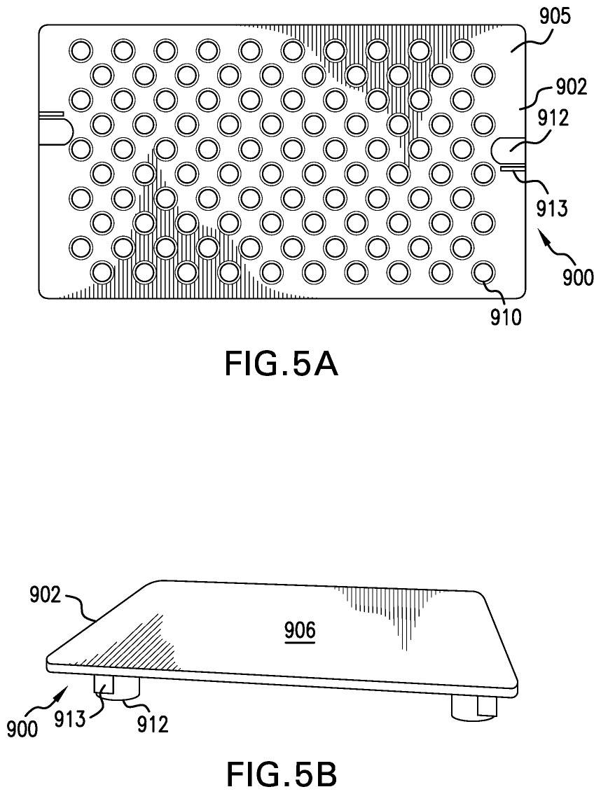

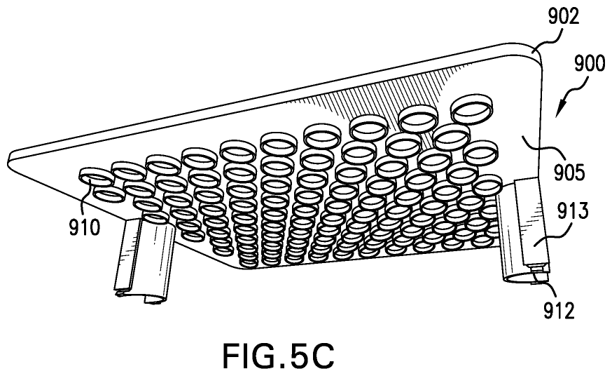

In some embodiments, a retainer may be used to secure syringes with the plunger rod assembled with the barrel. FIGS. 5A-C illustrate an embodiment of one such retainer in which retainer 900 comprises a generally rectangular body 902, the body having a lower surface 905 and an upper surface 906. Upper surface 906 may be flat. A plurality of thumb press receivers 910 may be formed on the lower surface 905 of the body. Each receiver 910 may be of different shape, such as a circular receiver, and formed as a depression or indentation in lower surface 905, or may include a protruding circumferential rim as best shown in FIG. 5C.

In some embodiments, receivers 910 may have an inner diameter or length that is slightly larger than the diameter or length of plunger thumb press 117. In at least some embodiments, receivers 910 may have an inner diameter of between ¼ inch and 2 inches. The receivers 910 may be disposed in rows and columns and arranged in any of the layouts and configurations described above with regard to the projections (e.g., rows and/or columns may be offset). Retainer 900 may further include a pair of arms 912 that extend from the lower surface 905. In some embodiments, arms 912 may be between 1 inch and 5 inches, or more specifically, between 2 inches and 5 inches, in length. Each of the arms 912 may be disposed next to a flange 913 that extends parallel to the arm 912, the arm 912 and the flange 913 being configured to friction fit within a tub.

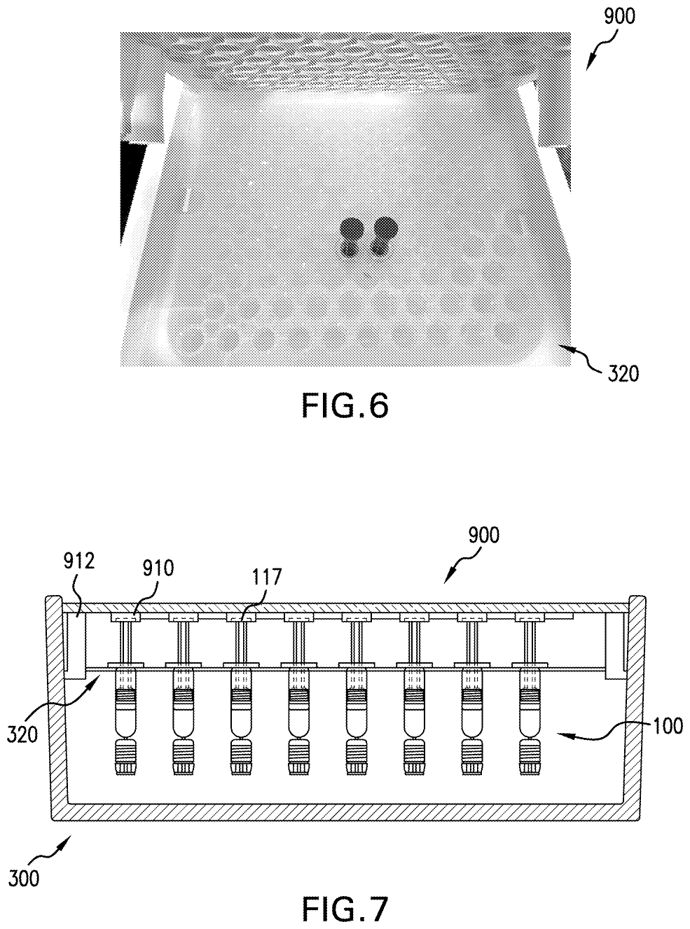

In use, syringes with both the barrel and the plunger rod assembled together may be disposed in a tray 320 (FIG. 6). For the sake of clarity, only a first row of syringes is shown in FIG. 7, although it will be understood that multiple rows of syringes may be disposed in a tub. As shown, tub 300 houses a tray 320 having a plurality of syringes 100. Each syringe is disposed with the plunger rod 110 being coupled to a stopper 130 and facing upward, and each thumb press 117 of plunger rod 110 may be disposed within a receiver 910 of retainer 900. Arms 912 may serve to secure the retainer 900 to tub 300 and/or tray 320, and to space the retainer 900 away from the tray to maintain the proper spacing between the receivers 910 and the thumb press 117. In at least some embodiments, arms 912 pass through cutouts of the tray and coupled thereto. With the thumb press 117 of plunger rods 110 disposed in the receivers 910, the plunger rods may impede movement of the stoppers. Specifically, when the external pressure decreases, the gas inside the barrel will attempt to expand, but the plunger rod 110 will prevent the stopper 130 from moving upward, the retainer 900 itself serving as a backstop for the plunger rod 110.

It is to be understood that the embodiments described herein are merely illustrative of the principles and applications of the present disclosure. For example, the number and arrangement of projections or receivers may be varied. Moreover, certain components are optional, and the disclosure contemplates various configurations and combinations of the elements disclosed herein. It is therefore to be understood that numerous modifications may be made to the illustrative embodiments and that other arrangements may be devised without departing from the spirit and scope of the present disclosure as defined by the appended claims.

It will be appreciated that the various dependent claims and the features set forth therein can be combined in different ways than presented in the initial claims. It will also be appreciated that the features described in connection with individual embodiments may be shared with others of the described embodiments.

What is claimed is:

1. A stopper retainer, comprising:
a base having an upper surface, a lower surface, and a perimeter;
a sidewall joined to the base around the perimeter, the sidewall having a raised height to define an interior cavity with the base; and
a plurality of projections extending from the lower surface of the base, each of the plurality of projections being sized to at least partially fit within a lumen of a syringe barrel.

2. The stopper retainer of claim 1, further comprising a plurality of depressions formed in the upper surface of the base and disposed in rows and columns.

3. The stopper retainer of claim 2, wherein the plurality of projections comprises a first number of projections, and the plurality of depressions comprises a second number of depressions, the first number and the second number being equal.

4. The stopper retainer of claim 3, wherein each of the plurality of depressions is aligned with one of the plurality of projections.

5. The stopper retainer of claim 4, wherein the plurality of projections is sized to at least partially fit within the plurality of depressions.

6. A system of storing syringes comprising:
a tub having an interior void;
a first tray having a plurality of apertures; and
the stopper retainer of claim 1;
wherein each of the apertures of the first tray is directly aligned with one of the plurality of projections of the stopper retainer.

7. The system of claim 6, further comprising a second tub and a second tray having a plurality of second apertures, wherein the second tub and the second tray being disposed on top of the stopper retainer.

8. The system of claim 7, further comprising a second stopper retainer, wherein each of the apertures of the second tray are directly aligned with one of the plurality of projections of the second stopper retainer.

9. The system of claim 7, further comprising 1-100 additional tubs and trays, and 1-100 additional stopper retainers, wherein each of the apertures of each additional tray are directly aligned with one of the plurality of projections of the additional stopper retainer immediately on top of the additional tray.

10. A stopper retainer, comprising:
a body having an upper surface and a lower surface; and
a plurality of receivers disposed on the lower surface of the body, each of the plurality of receivers being sized to fit a portion of a plunger rod thumb press.

11. The stopper retainer of claim 10, wherein each of the plurality of receivers is circular, square or irregular in shape.

12. The stopper retainer of claim 10, wherein each of the plurality of receivers comprises a circular, square or irregular shaped indentation in the body.

13. The stopper retainer of claim 10, wherein each of the plurality of receivers comprises a protruding circular, square or irregular shaped rim on the body.

14. The stopper retainer of claim 10, further comprising at least one arm extending from the lower surface of the body.

15. A system of storing syringes comprising:
a tub having an interior void;
a first tray having a plurality of apertures; and
the stopper retainer of claim 10;
wherein each of the apertures of the first tray is directly aligned with one of the plurality of receivers of the stopper retainer.

16. The system of claim 15, further comprising a plurality of syringes, each of the plurality of syringes comprising a barrel and a plunger rod, and each plunger rod being at least partially disposed in one of the plurality of receivers.
